(19) United States
(12) Patent Application Publication
(10) Pub. No.: US 2006/0204454 A1
(43) Pub. Date: Sep. 14, 2006

(54) METHOD FOR IMPROVING THE FUNCTIONAL PROPERTIES OF A GLOBULAR PROTEIN, PROTEIN THUS PREPARED, USE THEREOF AND PRODUCTS CONTAINING THE PROTEIN

(76) Inventor: Cecile Veerman, WAGENINGEN (NL)

Correspondence Address: THE WEBB LAW FIRM, P.C., 700 KOPPERS BUILDING, 436 SEVENTH AVENUE, PITTSBURGH, PA, 15219, US

(21) Appl. No.: 10/536,713

(22) PCT Filed: Nov. 28, 2003
(86) PCT No.: PCT/EP03/13678

§ 371 Date: Apr. 3, 2006

(30) Foreign Application Priority Data
Nov. 29, 2002 (EP) 02080019.9

(51) Int. Cl. A61K 8/64; A61K 8/96; A23L 1/31; C07K 14/765; C07K 14/415; C09D 189/00

(52) U.S. Cl. 424/50; 424/70.14; 530/350; 530/370; 426/574; 106/124.2

(57) ABSTRACT

The invention relates to a method for improving the functional properties of globular proteins, comprising the steps of providing a solution of one or more globular proteins, in which solution the protein(s) is/are at least partially aggregated in fibrils; and performing one or more of the following steps in random order: increasing the pH; increasing the salt concentration; concentrating the solution; and changing the solvent quality of the solution. Preferably, the solution of the one or more globular protein is provided by heating at a low pH or the addition of a denaturing agent. The invention also relates to the protein additive thus obtained, to the use thereof for food and non-food applications and to the food and non-food products containing the protein additive.

The invention relates to a method for improving the functional properties of a globular protein. The invention further relates to the protein thus prepared, to the use thereof in various products as a protein additive, in particular as a thickening agent, foaming agent, viscosity enhancing agent and/or gelling agent and to the products comprising such additive.

Food and non-food additives are inter alia concerned with improving and maintaining product quality. They are for example used to provide texture, consistency and stability. For this they have functional properties such as foaming properties, gelling properties, emulsifying properties, thickening properties etc.

For food applications, additives can be roughly divided into two groups, polysaccharides and proteins. Examples of the first group having thickening properties are e.g. guar gum, xanthan gum, locust bean gum. Examples of the second group are e.g. milk proteins. Among the milk proteins, whey proteins are widely used as ingredients in food products for their ability to form gels.

β-Lactoglobulin is the major protein component of the whey protein from milk. It is a globular protein with a molar mass of 18.3 kDa and a diameter of about 2 nm. When the protein is dissolved in an aqueous solution above a certain critical concentration and heated above the denaturation temperature (about 78° C.) it forms a gel. The globular structure unfolds at least partially and aggregates are formed. The gel is formed by heat treatment if the concentration of the protein is above a critical value (C_{P}), and an appropriate ionic strength is applied.

Polysaccharides have the advantage that they are effective thickeners in food products, even in low amounts. However, the price of these hydrocolloids is normally high. Moreover, at elevated concentrations they may often give rise to taste defects. When used in dairy products like desserts, they are considered non-natural.

Proteins are normally less effective (on a w/w basis) in thickening compared to hydrocolloids. Thus, even though their price may be considerably lower than for hydrocolloids, the higher dose required abolishes the price advantage.

As explained above, globular proteins form a gel when heated at neutral pH (around 7). However, the concentration needed to form the gel is relatively high, e.g. more than 5% (w/w). Moreover, a gel thus obtained is irreversibly formed and is therefore not suitable for use as thickener in a range of products. The gel would have to be dried and/or comminuted thus losing its thickening capacity. On the other hand, if whey proteins are thermally modified at neutral pH and low concentrations to avoid the undesired gel formation, the thickening capacity is very poor or not present at all.

In general there is a desire in the food industry to avoid additives that are non-natural. Proteins are a potential natural source for the preparation of additives but their functional properties are often not comparable to the presently used additives.

There is thus a need for proteins that have good functional properties, in particular thickening, gelling, foaming and emulsifying properties, and that are preferably highly effective at low concentrations.

In the research that led to the present invention it was found for β-lactoglobulin that the structures obtained at low pH confer to the solution containing them a much higher viscosity and have thus a higher gelling capacity than the structures formed by heating β-lactoglobulin at pH 7. Gelling agents of such low pH are however not practically useful.

When a solution of β-lactoglobulin is heated at a pH of about 2, denaturation leads to a different type of aggregation than at neutral pH. This low-pH denaturation leads to protein aggregates which are joined by physical forces, whereas denaturation at a pH around 7 or higher will lead to aggregates which are covalently bound through disulfide bonds.

It was found that heating a solution of β-lactoglobulin at a pH around 2 leads to formation of fibrillar protein structures. As stated above, it is generally accepted that these fibrils are constituted by aggregates held together by physical forces. The skilled person would expect that fibrils thus formed would decompose again upon pH increase.

In the research that led to the present invention it was surprisingly found that these fibrils are irreversibly formed when the heating time at or above denaturation temperature is longer than 10 minutes. The same observations were made for whey protein isolates and the teaching of the invention is thus applicable to globular proteins in general and to β-lactoglobulin and whey protein isolates and concentrates in particular.

It was furthermore found that similar fibrillar protein structures can be obtained when a denaturing agent is added to the solution comprising the globular protein.

The invention thus relates to a method comprising the steps of:

a) providing a solution of one or more globular proteins, in which solution the protein is at least partially aggregated in fibrils; and

b) performing one or more of the following steps in random order:
i) adjusting the pH of the solution to about neutral;
ii) increasing the salt concentration in the solution;
ii) concentrating the solution;
iii) changing the solvent quality of the solution.

In this way a protein additive is obtained having improved functional properties. Method step a) provides the fibrillar structures in the protein solution whereas method step b) triggers the protein such that it is ready to perform its function as a foaming, thickening, gelling or emulsifying agent upon addition thereof to the final product.

Providing a solution of the one or more globular proteins, in which solution the one or more proteins are at least partially aggregated in fibrils, can be achieved in various ways. In a first embodiment the fibril-containing solution of the one or more globular proteins is provided by heating a solution of the protein above room temperature, preferably at a temperature between 50 and 100° C., at a pH between 0.5 and 4, preferably between 0.5 and 3. In an alternative embodiment the fibril-containing solution of the one or more globular proteins is provided by adding a denaturing agent to the solution.

The denaturing agent can be a hydrotropic or chaotropic agent and is for example selected from the group consisting of ureum, guanidinium chloride, alcohols, such as methanol, ethanol, propanol, butanol, trifluorethanol. The treatment with the denaturing agent can be performed at a pH between 0.5 and 14, preferably between 3 and 11, more preferably between 5 and 9.

In solutions containing globular proteins that are treated in this way, fibrils are formed having an unexpectedly high gelling and/or thickening and/or foaming and/or emulsifying capacity. The fibrils are irreversibly formed and can be used at any desired pH or ionic strength.

Heating the solution in the first embodiment of step a) is preferably performed during at least 10 minutes, preferably at least 1 hour, more preferably at least 6 hours, most preferably at least 8 hours.

The pH of the treatment of the first embodiment of step a) is preferably below 2.8, preferably below 2.5, more preferably below 2.2. Suitable acids for adjusting the pH to this value are food grade acids, such as hydrochloric acid, phosphoric acid, nitric acid or sulphuric acid.

The total heating time required to obtain the effect may be achieved by batch wise heating, continuous flow heating or a combination of subsequent heating steps, e.g. by means of circulating a solution through a heating system.

Optionally, the solution is cooled before performing one or more of steps i) to iii).

It is preferred to cool the solution to a temperature between the denaturation temperature and 20° C., preferably between the denaturation temperature and 5° C.

When the pH is increased this is preferably to a value between 3.9 and 9, preferably to about neutral pH. Most food applications have a neutral, near neutral or slightly acidic pH.

Advantageously, the salt concentration is increased to a maximum of 0.2 M, preferably to 0.1 M. The salt used for increasing the salt concentration is preferably the salt of a divalent ion, preferably calcium. It was found that by adding calcium the functional properties are further improved.

According to a preferred embodiment step i) is performed prior to step ii) because pH adjustment in dilute systems is easier to carry out.

Changing the solvent quality of the solution can be performed by removing the denaturing agent, for example by dilution or dialysis.

In a further embodiment of the invention the method further comprises addition of already formed fibrils to the solution of globular proteins prior to the heating step. It was found that by means of this so-called seeding the heating time could be reduced. It was furthermore found that an even lower critical gelling concentration (Cp) could be obtained in samples that had been seeded as compared to samples that were not seeded. Seeds for addition to the solution can be prepared in the same way as the protein of the invention.

In order to obtain a dry product which is more stable upon storage the method further comprises the step of drying the solution to obtain a dry product. It was found that upon reconstituting the protein additive of the invention from the powder obtained after drying the same or similar functional properties were obtained. It is practical when the drying comprises spray drying. The dry product is preferably a powder. Alternatively granulates can be envisaged.

Furthermore it is possible to dilute a gel obtained after concentrating the heated solution according to step b) iii) of the method to a less viscous product by addition of a pH 2 solution. The same applies to a solution treated according to step b) ii) by lowering the salt concentration again.

Advantageously, the globular protein is a protein that is substantially non-denatured and is capable of being thermally denatured at a temperature at or above the denaturation temperature of the protein or chemically denatured.

The method of the present invention can be performed with a wide variety of globular proteins, such as whey proteins, egg albumins, blood globulins, soy proteins, wheat proteins, in particular prolamines, potato proteins or pea proteins. In a preferred embodiment, the globular protein is a whey protein isolate or a whey protein concentrate, preferably a whey protein concentrate enriched in (e.g. >40%) β-lactoglobulin. In a much preferred embodiment the globular protein is β-lactoglobulin.

In a further embodiment the globular protein is the whey protein isolate powder (95% protein, w/w) that is commercially available under the name Bipro™ and is composed of ˜70% β-lactoglobuling, ˜18% α-lactalbumin, ˜6% bovine serum albumin, and ˜6% immunoglobulins. The functional properties of this product after having been subjected to the method of the invention can be further improved by purifying the product prior to heating at low pH. Such purification comprises acidification to pH 4.75, centrifugation and use of the supernatant. This treatment results in loss of about 10% (aggregated) protein, mainly BSA.

The invention further relates to a protein additive for food and non-food applications based on a system of one or more proteins that are aggregated to form fibrils, characterized in that the protein additive has improved functional properties as compared to a similar protein additive based on a system of the same one or more proteins in the same concentration in which the proteins are not aggregated into fibrils. Fibrils in this respect are preferably fibrils consisting of protein and having an aspect ratio of 5 or higher. The aspect ratio is the ratio between length and width or length and height or length and diameter. The length of the fibrils is preferably equal to or above 100 Å and equal to or below 1 mm, preferably below 100 μm. These fibrils can be made visible by means of a microscope.

The above described protein additive can be obtained by the method of the invention or by any other means that leads to the above described structural properties.

The protein additive of the invention can be used as a stabilizer of foams, dispersions and emulsions. Foams are systems of a gas in a liquid. Emulsions are liquids in liquids and dispersions are solids in liquids. Usually these systems cannot exist without the help of a stabilising agent that helps in maintaining the disperse phase uniformly distributed in the continuous phase. The protein additive of the invention was found to be very suitable for this purpose.

The protein additive can be used in food stuffs, such as dairy products, for example (aerated) desserts, yogurts, flans, in bakery or confectionary applications, such as frappe, meringue, marshmallows, in cream liqueurs or in beverage foamers, such as cappuccino foamers. When using β-lactoglobulin, whey protein concentrate or whey protein isolate as the globular protein that constitutes the protein additive the product obtained can be an all milk product.

Whey protein concentrates normally comprise 25-90% (w/w) whey protein. Whey protein isolates usually comprise >90% whey protein.

The protein additive of the invention can also be used in meat products, e.g. comminuted meat products (Frankfurter sausages), hamburgers, luncheon meat, pâte's, poultry, fish meat products or meat replacers on vegetable basis, to enhance the water-binding and/or texture of the product.

Alternative applications of the protein additive of the invention can be found in non-food products such as paints, cosmetics, toothpastes, deodorants etc.

The invention further relates to products comprising the protein additive of the invention, such as food stuffs, in particular dairy products or meat products, but also non-food products, e.g. paints, cosmetics, toothpastes, deodorants.

According to a further aspect thereof the invention relates to a protein composition comprising one or more particles having texturizing properties, wherein the protein molecules are aggregated into fibrils. Texturizing properties comprise the ability to promote or modify the viscosity or gelling ability of a product containing the composition. Preferably, the fibrils have an aspect ratio, which is defined as the ratio between length and width or length and height or length and diameter, of 5 or higher. The length of the fibrils is preferably equal to or above 100 Å and equal to or below 1 mm, preferably below 100 μm.

The protein additive of the invention has improved functional properties. Functional properties comprise thickening capacity, gelling capacity, foaming capacity and emulsifying capacity and all have to do with the structure and texture of the product containing the additive. The fact that the functional properties of the additive are improved means that the capacity to induce gelling, foaming, thickening or emulsification in the product containing the protein additive is improved as compared to the capacity to do so of the same protein in the same concentration but which is not subjected to the method of the invention.

The present invention will be further illustrated in the examples that follow and that are not intended to limit the invention. In the Examples reference is made to the following figures.

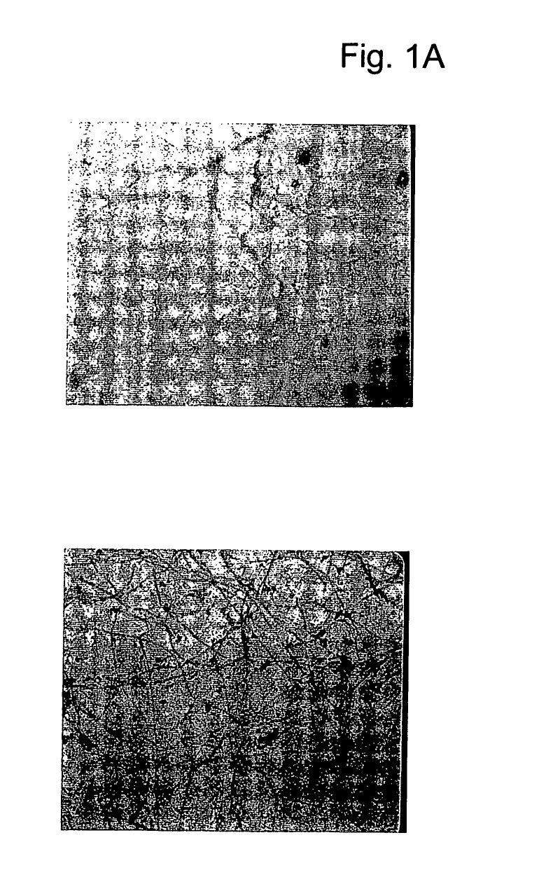

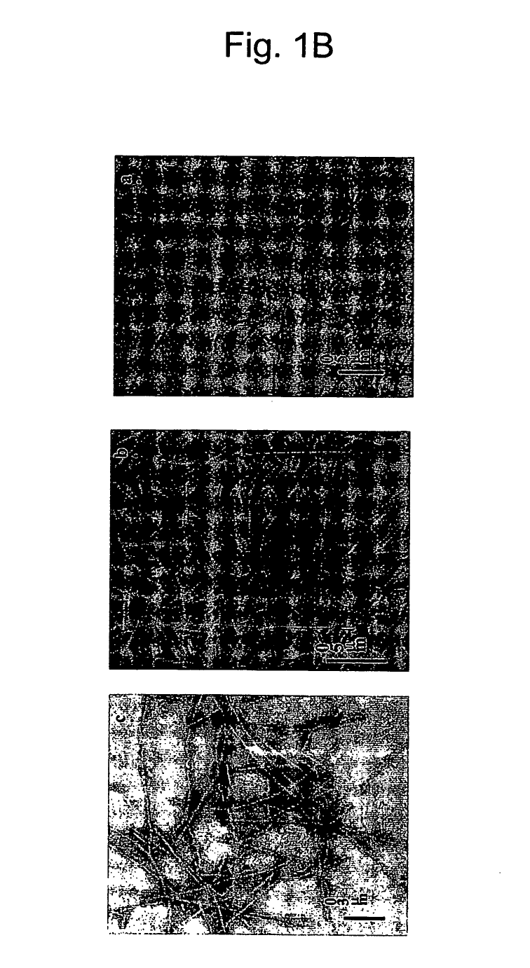

FIG. 1A shows a TEM photograph of Bipro™ treated according to the invention after different heating times.

FIG. 1B shows TEM photographs of β-lactoglobulin treated according to the invention after neutralization to different pHs.

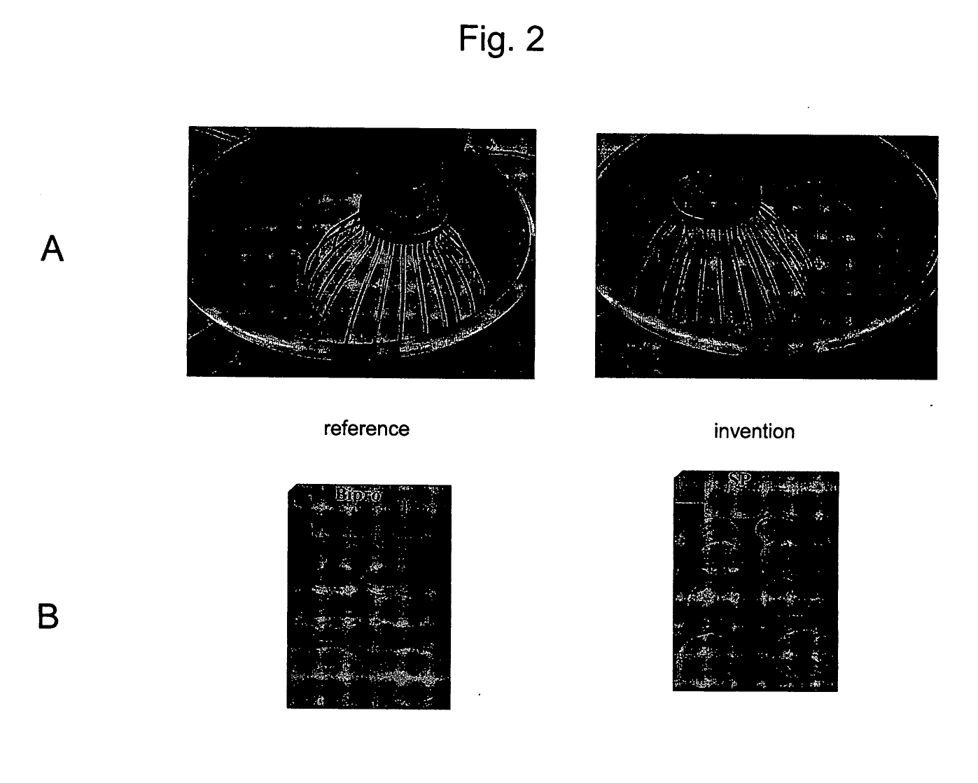

FIG. 2A shows meringue foam of treated and untreated Bipro™ prior to drying.

FIG. 2B shows meringue foam of treated and untreated Bipro™ after drying.

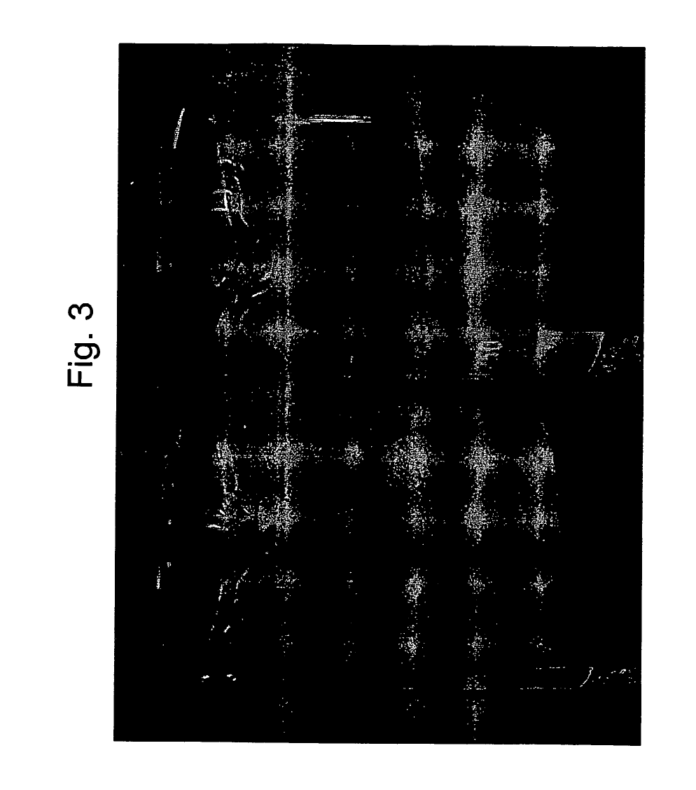

FIG. 3 shows cappuccino foam prepared with treated and untreated Bipro™.

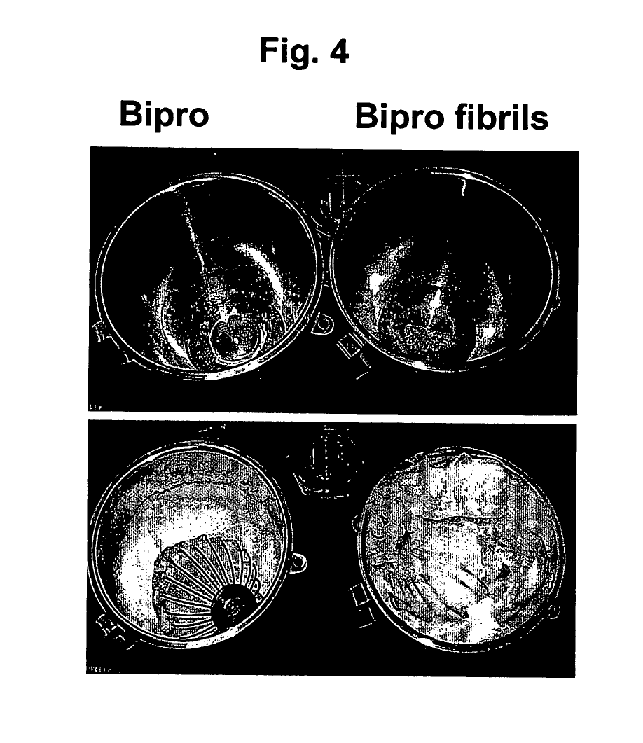

FIG. 4 shows the overrun of a foam prepared with treated and untreated Bipro™.

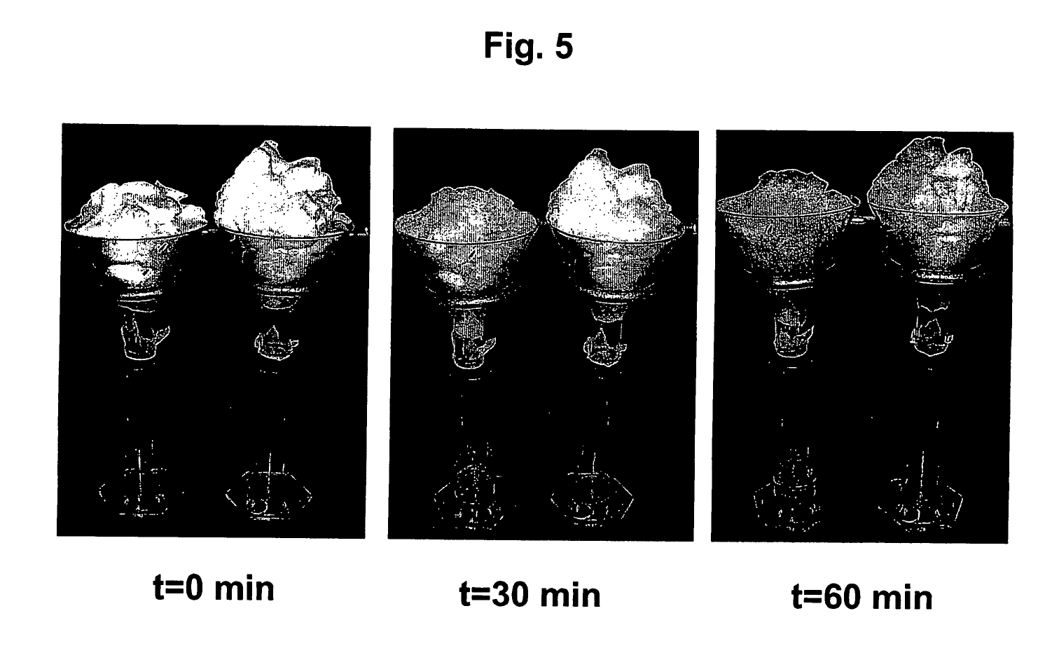

FIG. 5 shows the foam stability in time of a product prepared with treated and untreated Bipro™.

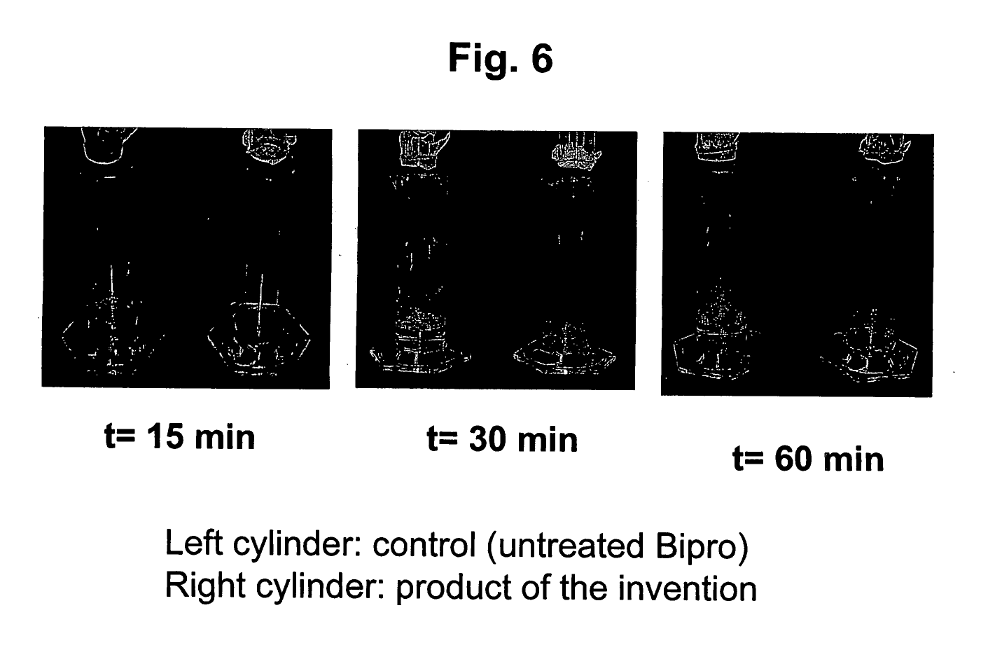

FIG. 6 shows the drainage in time of a foam prepared with treated and untreated Bipro™.

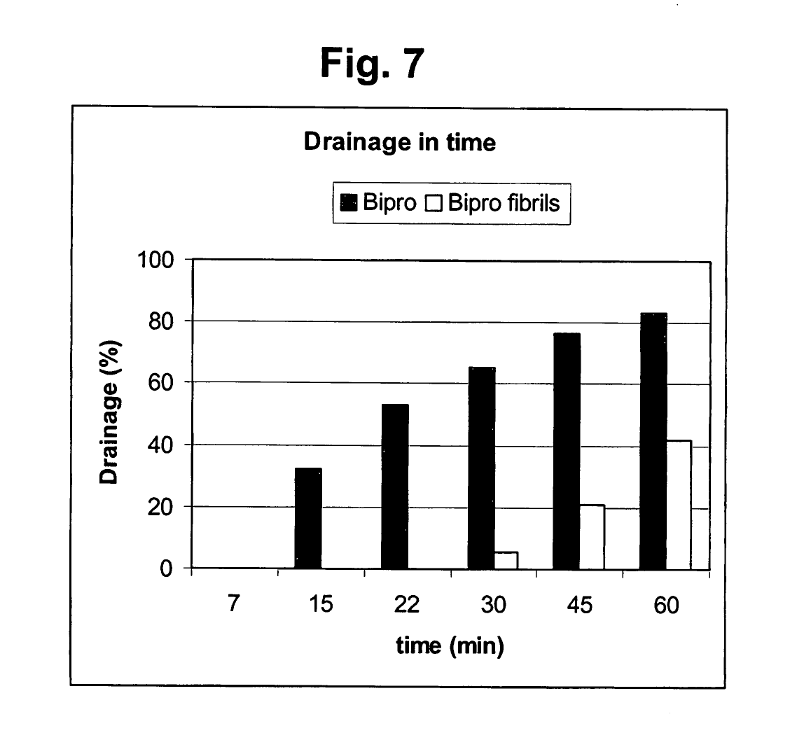

FIG. 7 shows the drainage in time of a foam prepared with treated and untreated Bipro™.

### EXAMPLES

### Example 1

### Preparation of β-Lactoglobulin Gels According to the Invention, and Determination of Critical Gelling Concentration

β-Lactoglobulin (β-lg) was obtained from Sigma (L-0130) and is a mixture of the genetic variants A and B. The protein was dissolved (3% w/w) in a HCl solution at pH 2. To remove traces of calcium ions from the β-lg, and to obtain a protein solution with the same pH and ionic strength as the solvent, the protein was diluted repeatedly with HCl solvent and filtered through a 3K filter in an Omegacell™ membrane cell (Filtron) at 4° C. and a maximum pressure of 3 bar. The procedure was stopped, when the pH and conductivity of the diluted solution and the solvent were the same.

The β-lg solution was centrifuged at 22600 g for 30 min. To remove any traces of undissolved protein, the supernatant was filtered through a protein filter (FP 030/2, 0.45 mm, Schleicher & Schuell). A UV spectrophotometer was used to determine the β-lg concentration at a wavelength of 278 nm.

β-Lactoglobulin (w/w) as prepared above diluted to a concentration of 2% was heated at 80° C. for 10 h in a water bath. After cooling, the pH was adjusted to pH 7 or 8 with 0.1 and 1 M NaOH. Various CaCl₂ concentrations (0.005, 0.0075, 0.01, 0.05, and 0.1 M) were added very carefully on ice, and the solution was mixed well. After this procedure, the solution was poured into the VOR rheometer (Bohlin concentric cylinder geometry C14) to determine the critical gelling concentration. The sample in the VOR was heated from 3° C. to 25° C. After 3 h in rest, a strain sweep was performed (frequency 1 Hz, temperature 25° C., strain 0.000206-0.206).

The procedure was repeated for various protein concentrations. To determine the critical gelling concentration Cp, first the G′ (the “elastic modulus”, a characteristic for the elastic component of a system) was determined for various protein concentrations (in the linear region of the curve). A plot was made of (G′)^{1/t} versus concentration c, for t ranging between 1.7 and 4.5. t is a scaling factor. In the fitting procedure, we make use of the physical fact that extrapolation of (G′)^{1/t} to zero should yield the same Cp for all t>0. The scaling assumption has the implication that when t is close to the correct value, the data points will be on a straight line. When t is larger than the correct value, the fit through the points will bend away from the straight line and will lie below it. When t is smaller than the correct value, the fit through the points will also bend away from the straight line but will now lie above it. In that case, the slope of the fit at the intercept with the horizontal axis will be zero. Therefore, in the fitting procedure we use the fact that the curvature of the fit will change if different values for t are chosen, while the intercept Cp will have to remain the same.

Cp was determined from fits through the data points that are closest to a straight line in determining an average intercept, Cp. It appeared that the Cp values for the protein system according to the invention were considerably lower than for the reference (not-modified) protein system. (see examples)

The results of this experiment are shown in Table 1.

### Example 2

### Preparation of β-lg Gels According to the Conventional (Neutral pH) Gelation Method, and Determination of the Critical Gelling Concentration

β-Lactoglobulin (β-lg) was obtained from Sigma (L-0130) and is a mixture of the genetic variants A and B. The protein was dissolved (3% w/w) in a HCl solution at pH 2. To remove traces of calcium ions from the β-lg, and to obtain a protein solution with the same pH and ionic strength as the solvent, the protein was diluted repeatedly with HCl solvent and filtered through a 3K filter in an Omegacell™ membrane cell (Filtron) at 4° C. and a maximum pressure of 3 bar. The procedure was stopped, when the pH and conductivity of the diluted solution and the solvent were the same.

The β-lg solution was centrifuged at 22600 g for 30 min. To remove any traces of undissolved protein, the supernatant was filtered through a protein filter (FP 030/2, 0.45 mm, Schleicher & Schuell). A UV spectrophotometer was used to determine the β-lg concentration at a wavelength of 278 nm.

3% β-lg samples at pH 7 or 8 were heated at 80° C. for 30 min. After cooling, 0.01 M CaCl₂ was added very carefully on ice, and the solution was mixed well. After this procedure, the solution was poured in the VOR (Bohlin concentric cylinder geometry C14). The sample in the VOR was heated from 3° C. to 25° C. After 3 h in rest, a strain sweep was performed (frequency 1 Hz, temperature 25° C., strain 0.000206-0.206). Subsequently the critical gelling concentration of the conventionally formed β-lactoglobulin gel was measured. The results are shown in Table 1.

TABLE 1
Determination of the Critical gelling concentration
(gels prepared according to examples 1 and 2)

| | | | | Critical gelling |
|---|---|---|---|---|
| | Example | Final | [mM] | concentration |
| Heating conditions | no.: | pH | CaCl₂ | (% w/w) |
| pH 2, 10 hrs, 80° C. | 1 | 7.0 | 0 | 1.3 |
| pH 2, 10 hrs, 80° C. | 1 | 7.0 | 5 | 1.1 |
| pH 2, 10 hrs, 80° C. | 1 | 7.0 | 7.5 | 1.0 |
| pH 2, 10 hrs, 80° C. | 1 | 7.0 | 10 | 0.1 |
| pH 2, 10 hrs, 80° C. | 1 | 7.0 | 50 | 0.6 |
| pH 2, 10 hrs, 80° C. | 1 | 7.0 | 100 | 0.7 |
| pH 2, 10 hrs, 80° C. | 1 | 8.0 | 10 | 0.4 |
| pH 2, 10 hrs, 80° C. | 1 | 8.0 | 50 | 0.6 |
| pH 2, 10 hrs, 80° C. | 1 | 8.0 | 100 | 0.9 |
| pH 7, 0.5 hrs, 80° C. | 2 | 7.0 | 10 | No gel |
| | | | | formed at 3% |
| pH 7, 0.5 hrs, 80° C. | 2 | 8.0 | 10 | No gel |
| | | | | formed at 3% |

The results show that β-lactoglobulin modified by the acid pretreatment has a higher gelling ability than β-lactoglobulin which is not acid-modified.

### Example 3

### Modification of Bipro™

Bipro™, a whey protein isolate powder (95% protein, w/w), was obtained from Davisco, USA. Besides β-lactoglobulin, Bipro™ also contains α-lactalbumin, bovine serum albumin and immunoglobulines.

Modification of Bipro™ was carried out as follows: Four Bipro™ solutions were prepared in demineralised water in concentrations of 3, 4, 5 and 6% w/w. The pH was adjusted to pH 2, using HCl. The solutions were heated for 10 hours at 80° C. After cooling, the samples were neutralised with NaOH to pH 7, and cooled further to 3° C., after which CaCl₂ (5 mM) was added to half of the samples. After 3 hours, all samples were assessed visually. The results are given in table 2.

A control experiment was carried out in the following way. Bipro™ solutions in demineralised water were made (3, 4, 5, 6% w/w) having a pH of 7. The solutions were heated at 80° C. for 10 hrs, then cooled to 3° C. and CaCl₂ was added to half of the samples. After 3 hours, the samples were assessed visually. The results are shown in Table 2.

TABLE 2
Visual rheological properties of modified and not-modified
Bipro ™ (from Example 3)

| Bipro ™ samples: | no CaCl₂ added | 5 mM CaCl₂ added |
|---|---|---|
| pH 2 modified | | |
| and neutralised: | | |
| (% w/w) | | |
| 3 | Viscous solution | Gel |
| 4 | Very viscous sol. | Gel |
| 5 | Very viscous sol. | Firm gel |
| 6 | Very viscous sol. | Very firm gel |
| Not modified: | | |
| 3 | Low viscous liquid | Liquid |
| 4 | Low viscous liquid | Liquid |
| 5 | Low viscous liquid | Liquid |
| 6 | Low viscous liquid | Liquid |

From the table it clearly follows that treatment according to the invention, of a whey protein product comprising different types of protein, also leads to strongly enhanced gelling capacity and a strong increase in viscosity.

### Example 4

### Effect of Seeding

Introduction

The objective of this example was to study the effect of addition of seeds to fresh protein material prior to heating at pH 2. The total protein concentrations, the ratios between fresh protein material and seeds (fresh/seeds), and the heating time of both seeds and the mixtures of fresh and seeds were varied. The total protein concentration at which seeds were made was kept constant, in order to have the same seeds in the different experiments. The protein material was Bipro™, a whey protein isolate powder (95% protein, w/w).

Materials and Methods

Bipro™ was obtained from Davisco, and is composed of ˜70% β-lactoglobuling, ˜18% α-lactalbumin, ˜6% bovine serum albumin, and ˜6% immunoglobulins. The protein powder was dissolved in NANOpure™ water and left to stir at room temperature for 3 hours. Next the pH was adjusted to pH 4.75, using 6 M HCl. The protein solution was centrifuged at 12000 rpm for 30 min at room temperature, using a SLA-1500 super lite aluminium rotor in the Sorvall RC-5B refrigerated superspeed centrifuge. At pH 4.75, which is close to the iso-electric point, undissolved protein is precipitated. HPLC analysis shows that about 50% of the BSA present in Bipro™ is being removed in this centrifugation step, indicating that the BSA was aggregated. To remove any traces of undissolved protein that did not end up in the pellet the supernatant was filtered through a protein filter (FD 30/0.45 mm Ca—S from Schleicher & Schuell). The centrifugation step at pH 4.75 is further referred to as “purification”, meaning removal of aggregated and undissolved material, and the material is called “purified Bipro™”.

After centrifugation and filtration the pH of the Bipro™ solution was set at pH 2, using 6 M HCl. The protein concentration was determined using a UV spectrophotometer and a calibration curve of known protein concentrations at wavelength 278 nm.

A Bipro™ stock solution of 1.2% (w/w) at pH 2 was prepared according to the method described above. Different samples were taken and heated for 2, 5, or 10 h at 80° C. After heating the samples were cooled and stored in a refrigerator. Part of each sample was diluted to 0.8 and 0.4% Bipro™. Also the unheated Bipro™ solution was diluted to 0.8 and 0.4% Bipro™. These “stock” solutions of unheated (fresh) and heated material (seeds) after different heating times were mixed in different ratios and heated for different times at pH 2 and 80° C.

Cold gelation experiments with seeds made at 1.2% Bipro™ were performed as follows. Seeds that were made by heating Bipro™ from the 1.2% stock solution were used after dilution with NANOpure™ adjusted to pH 2 with 6M Hcl to the required total concentrations. The total Bipro™ concentrations studied for this batch were 0.4%, 0.8%, 1.0%, and 1.2% Bipro™. Unheated and heated material were mixed in different ratios (0% seeds, 10% seeds, 20% seeds, 70% seeds, and 90% seeds) and these mixtures were heated for 10 h, at pH 2 and 80° C. After heating, the samples were cooled and set at pH 7, using 1.0 M and 0.1 M NaOH.

Another set of cold gelation experiments was done, but this time the seeds used were prepared at a Bipro™ concentration of 2.0%. In order to be able to go to higher total protein concentrations when mixing seeds and fresh, and also using the same seeds for those different total protein concentrations, a higher concentration for preparing the seeds was needed. The total Bipro™ concentrations studied for this set were 0.8%, 1.0%, 1.2%, 1.4%, and 1.6% Bipro™. Also here fresh and seeds were mixed in different ratios and heated for 10 h. at pH 2 and 80° C. After heating, the samples were cooled and set at pH 7, using 1.0 M and 0.1 M NaOH.

In order to see the effect of addition of seeds of non-dialysed, purified Bipro™, a series of test tubes was filled. The mixtures (at pH 2) were heated for either 2, 5, or 10 hours at 80° C. After cooling the samples to room temperature and overnight storage the tubes were visually examined. All gelation experiments were performed at 10 mM CaCl₂.

The samples that were heated in the presence of seeds and subsequently cooled and set at pH 7 were cooled on ice prior to addition of CaCl₂, in order to slow down the reaction rate of cold gel formation upon addition of calcium. A Paar Physica MCR 300 stress controlled rheometer with a concentric cylinder geometry (CC10) was used. The rheometer was cooled to 3° C. before the sample was put into the geometry. The rheometer was heated to 25° C. After 3 h of rest at 25° C. a strain sweep was performed (frequency 1 Hz, temperature 25° C., strain 0.001-1).

Results

Mixtures of seeds and fresh Bipro™ of different concentrations (%) (w/w) were heated for 2, 5, or 10 h at pH 2 at 80° C. After cooling of the samples they were visually examined. It was found that upon the addition of seeds, gelation occurs at a lower total protein concentrations than when no seeds are present. Furthermore it was found that for a higher total protein concentration a higher G′ is observed. When a higher amount of seeds is present during heating, a higher G′ is observed and when the added seeds were heated for a longer time, the resulting G′ after mixing with fresh and heating again is higher than for shorter heating time of the seeds. In addition, longer heating of the mixtures of fresh and seeds result in higher G′.

When plotting the graphs for different total Bipro™ concentrations for a certain amount of seeds present upon heating, Cp values per seeds-concentration were determined. From the linear regime of the strain sweep curves (i.e. where the G′ is independent of the strain), G′ was determined. The method to determine Cp and t is described by Van der Linden and Sagis, Langmuir 17, 5821 (2001), which is the same method as in Example 1.

The resulting Cp and t values for the different amount of seeds present during heating of the samples are given in Table 3.

TABLE 3
Calculated values for Cp and t

| Amount of seeds | Cp | t |
|---|---|---|
| 0% | 0.58% ± 0.12 | 1.91 ± 0.33 |
| 10% | 0.53% ± 0.03 | 1.84 ± 0.12 |
| 20% | 0.20% ± 0.12 | 1.84 ± 0.30 |
| 70% | 0.18% ± 0.10 | 1.81 ± 0.20 |
| 90% | 0.57% ± 0.10 | 2.17 ± 0.35 |

From Table 3 it can be concluded that the critical percolation concentration (also known as critical gelling concentration) is decreased due to the presence of seeds. This means that less protein is needed for obtaining the same result.

### Example 5

### Transmission Electron Microscopy

Bipro™

TEM micrographs were made in order to obtain insight in the structures formed upon heating the Bipro™ samples for different heating times and to see whether there are differences between samples. The samples (heated at 1.2% Bipro™ at pH 2) were diluted to 0.05%. The TEM samples were prepared by negative staining. A drop of the diluted solution was deposited onto a carbon support film on a copper grid. The excess was removed after 15 s using a piece of filter paper. A droplet of 2% PTA (pH 5.5) was added for 15 s, any excess being removed with filter paper. The grid was left to dry to the air. Electron micrographs were made using a Philips CM 12 Transmission Electron Microscope operating at 80 kV. The sample that was heated for 2 h did not show fibrils. In the samples that were heated for either 5 or 10 h long fibrils were visible (see FIG. 1A).

β-lactoglobulin

Transmission Electron Microscope (TEM) photographs were made of the samples after heat treatment at pH 2, and of samples that were neutralized at pH 7 and 8 (see FIG. 1B). From this it follows that the fibrils do not disintegrate upon neutralisation.

Description of the Sample Preparation for TEM:

The following samples:

a) 2 % beta-lactoglobuline, pH 2, 10 hrs 80° C.
b) as a), but neutralized to pH 7 using 1.0 and 0.1 M NaOH
c) as a), but neutralized to pH 8 using 1.0 and 0.1 M NaOH were diluted to 0.04% beta-lactoglobuline. The TEM samples were prepared by negative staining. A drop of the diluted solution was deposited onto a carbon support film on a copper grid. The excess was removed after 30 seconds using a piece of filter paper. A droplet of 2% uranyl acetate pH 3.8, was added for 15 seconds; any excess was removed again as before. Electron micrographs were made using a Philips CM 12 Transmission Electron Microscope operating at 80 kV.

### Example 6

### Effect of pH, Drying and Concentration on Overrun and Stability of Foam

Introduction

Various experiments have been performed in which foam properties of the fibrils formed are determined. In this the effect of pH, drying and concentration at which the fibrils are formed on the foam properties is tested.

Material and Methods

A Bipro™ solution is prepared and purified as follows. Bipro™ is solubilised in water in a concentration of 10, 12.5 en 15%. These solutions are acidified to pH 4.75 with 6M HCl by adding the HCl solution drop by drop under constant stirring. At pH 4.75 the Bipro™ solution turns white with large flakes which sediment slowly. The solution is centrifuged at 10 min, 9000 rpm in a Sorvall superspeed RC2-B centrifuge, GSA rotor (13.200 g). The clear supernatant is collected and spray dried at pH 4.75. The pellet is discarded.

The fibrils are formed by heating the purified Bipro™ solution at pH 2 (acidified with 6M HCl) during 10 hours. The solution is cooled down by gradually (0.5-1 hour) cooling the water bath from 80 to 20° C. The pH is increased by adding NaOH (2M) under stirring. The solution turns white between pH 4 and 5.5 and slowly becomes clear upon further increasing the pH.

Fibrils formed of purified Bipro™ are called 2-step fibrils and in case non-purified Bipro™ is used it is called 1-step fibrils.

Foam is obtained by whipping under standard conditions a 3% protein solution for 5 min at speed 3 in a Hobart mixer (model N-50) provided with a standard bowl and wire whisk. The foam is transferred to a round bottom bowl of stainless steel with a diameter of 10 cm, height 5.4 cm, a volume of 270 ml and a weight of 52.1 g.

The overrun and stability are measured as follows. For the overrun the round bottom bowl is weighed (A) and filled with foam. A spatula is used to straighten the surface and this bowl is weighed again (B). For the stability the foam is brought in a weighted powder funnel (D) and the filled funnel (C0) is weighed. The funnel is brought above the cylinder and the cylinder (Wt) and funnel (Et) are weighed after 15, 30, 45 and 60 min.

The overrun and stability (drainage) are calculated as follows.
Overrun (%)=(V*S/(B−A)*100)−100
V=volume round bottom bowl
S=specific weight protein solution
B=weight bowl and foam
A=weight bowl
   Stability (%)=(((C−D)−(C−E))/(C−D))*100
C=funnel+foam after filling
D=weight empty funnel
Et=weight funnel after 15, 30 45 or 60 min drainage.
   Drainage (%)=Wt/(C−D)*100
Wt=weight cylinder after 15, 30, 45 or 60 min drainage
C=weight funnel and foam after filling
D=weight empty funnel.
   Results and Discussion
   Effect of pH

In Table 4 the results of the foam tests of native Bipro™ and 2-step fibrils are shown. The results show that whipping at pH 7 gives a high overrun and a 76% drainage in 60 min. Whipping of the same fibrils at pH 5 gives 50% lower overrun but only 32% drainage in 60 min. As a comparison purified Bipro™ is whipped and this gives a low overrun and a high drainage.

TABLE 4

| | modifi- | | whipping | | | | | | |
|---|---|---|---|---|---|---|---|---|---|
| | cation | | test | | over- | drainage | drainage | drainage | drainage |
| | % | | % | | run | % | % | % | % |
| code | Bipro | pH | Bipro | pH | % | (15 min) | (30 min) | (45 min) | (60 min) |
| pH 7 | 4 | 2 | 3 | 7 | 3599 | 2 | 35 | 61 | 76 |
| fibrils | | | | | | | | | |
| pH 6 | 4 | 2 | 3 | 6 | 2713 | 0 | 9 | 33 | 54 |
| fibrils | | | | | | | | | |
| pH 5 | 4 | 2 | 3 | 5 | 1737 | 0 | 6 | 20 | 32 |
| fibrils | | | | | | | | | |
| pH 7 | 4 | 2 | 3 | 7 | 1676 | 29 | 70 | 84 | 90 |
| native | | | | | | | | | |
| pH 6 | 4 | 2 | 3 | 6 | 1306 | 20 | 60 | 74 | 81 |
| native | | | | | | | | | |
| pH 7 | 4 | 2 | 3 | 5 | 1525 | 8 | 46 | 64 | 73 |
| native | | | | | | | | | |

Effect of Concentration At Which the Fibrils are Formed

2-step Bipro™ fibrils are formed at 3-6% Bipro™ concentration. These solutions are diluted to 3% and whipped. The concentration at which the fibrils are made does hardly effect the overrun, the drainage seems somewhat smaller in case the fibrils are made at higher concentrations (Table 5).

TABLE 5

| | modifi- | | whipping | | | | | | |
|---|---|---|---|---|---|---|---|---|---|
| | cation | | test | | over- | drainage | drainage | drainage | drainage |
| | % | | % | | run | % | % | % | % |
| code | Bipro | pH | Bipro | pH | % | (15 min) | (30 min) | (45 min) | (60 min) |
| 3% fibrils | 3 | 2 | 3 | 7 | 3665 | 0 | 17 | 43 | 55 |
| 4% fibrils | 4 | 2 | 3 | 6 | 3773 | 0 | 13 | 34 | 53 |
| 5% fibrils | 5 | 2 | 3 | 5 | 3829 | 0 | 5 | 26 | 43 |
| 6% fibrils | 6 | 2 | 3 | 7 | 3467 | 0 | 1 | 18 | 40 |

Effect of Drying

The whipping experiments are performed with 1-step fibrils. Additionally, there is salt added before heat treatment and salt is also present during whipping. In general addition of salt causes the formation of larger structures during heating and a better overrun and foam stability.

The results in Table 6 show that freeze drying hardly effects the overrun and the draiage. It also shows the poor foam properties of native Bipro™.

TABLE 6

| | modification | | | whipping test | | | | drainage | drainage | drainage | drainage |
|---|---|---|---|---|---|---|---|---|---|---|---|
| | % | | NaCl | % | | NaCl | overrun | % | % | % | % |
| code | Bipro | pH | mM | Bipro | pH | mM | % | (15 min) | (30 min) | (45 min) | (60 min) |
| freeze dried | 4 | 2 | 30 | 3 | 7 | 22.5 | 2159 | 0 | 20 | 39 | 57 |
| fibril powder | | | | | | | | | | | |
| fresh fibrils | 4 | 2 | 30 | 3 | 6 | 22.5 | 2278 | 0 | 16 | 43 | 59 |
| (prior to freeze | | | | | | | | | | | |
| drying) | | | | | | | | | | | |
| native | 4 | 2 | 30 | 3 | 5 | 30 | 464 | 74 | 86 | 91 | 92 |
| Bipro ™ | | | | | | | | | | | |

### Example 7

### Foaming Test

75 Grams of a 3% solution of purified Bipro™ and purified Bipro™ treated according to the invention were whipped in a Hobart N 50 mixer for 5 min at speed 3. The overrun of Bipro™ was 1600%, whereas the overrun of Bipro™ fibrils (i.e. Bipro™ treated according to the invention) was 3400%. When non-purified Bipro™ without fibrils was used as a starting product the overrun was only 450%. FIGS. 4-7 show the results. It follows that Bipro™ fibrils show a very high overrun. The foam drains in time, but drainage is much quicker for untreated Bipro™.

### Example 8

### Use of the Protein Additive of the Invention as a Thickening Agent in Custard-Like Cream Dessert

Modified Bipro™ was obtained by freeze-drying a sufficient amount of the neutralized 5% solution as described in Example 3. The powder thus obtained can be used directly in the applications below, or mixed with calcium chloride prior to use in the applications.

Composition:

| | A. traditional | B. invention |
|---|---|---|
| | (grams) | (grams) |
| Skim milk | 355 | 355 |
| Cream (40% fat) | 65 | 65 |
| Water | 444 | 444 |
| Protein: | | |
| Esprion 300U | 10 | — |
| (DMV International) | | |
| Modified Bipro ™ | — | 0.8 |
| Saccharose | 60 | 60 |
| Lactose | 28 | 37 |
| Modified starch | 38 | 38 |
| (C*tex 06201 from Cerestar) | | |
| Carrageenan | 0.3 | 0.3 |
| (CL 360C, Danisco) | | |
| Flavouring | q.s. | q.s. |
| (e.g. vanilla) | | |
| Colouring | q.s. | q.s. |

Esprion™ 300U is a whey protein concentrate having 30% protein (w/w).

All the ingredients were mixed in the cold milk (approx. 7° C.), and left to hydrate for 20 minutes at a temperature <10° C. The mixture was heated for 10-20 seconds at 140° C. using an UHT pasteuriser (APV, Denmark) fitted with a holding tube, and subsequently cooled to <10° C., and packaged. Storage was at a temperature below 10° C.

Products obtained are tested by a panel, and a texture measurement was carried out using the Stevens Texture Analyser™ (Stevens Instruments, UK) equipped with a disc probe. The resistance of the probe was measured as the probe penetrates the sample within a determined period of time over a specified distance.

The test results showed that, despite the low dosage of modified Bipro™, the texture of Sample B was much better (better mouth feel, higher viscosity) than sample A.

### Example 9

### Use of the Protein Additive of the Invention as a Thickening Agent in in Drinking Yogurt

Yogurt A (reference) was prepared as follows. 117 grams of Esprion™ 300U was dissolved in 1 liter of water. 280 Grams of this solution was mixed with 720 grams of skim milk. The final protein concentration of this solution was 3.5% (w/w). The solution was heated to 65° C. and homogenised at this temperature, after which it was pasteurised for 6 minutes at 92° C. The pasteurised milk was cooled to 32° C., and inoculated with a yogurt culture (0.02% Yoflex™ 380 from Chr. Hansen). Fermentation was continued for approx. 14-16 hours until a pH of 4.2-4.3 was reached.

Drinking yogurt was prepared by blending the freshly prepared yogurt with a fruit preparation (25% water, 25% fruit juice, 50% sugar obtainable from Wild, Germany) in a ratio 80% yogurt/20% fruit preparation. Before adding to the yogurt, the fruit preparation was pasteurised at 85° C. for 5 minutes and cooled to 20° C.

The mixture of yogurt and fruit preparation was subjected to a low-pressure homogenisation at 1-3 MPa. The drinking yogurt was then cooled to <10° C., packaged and stored below 10° C.

Yogurt B including the protein preparation according to the invention was prepared in a similar way as A but the starting milk was composed of 280 grams of an 0.8% (w/w) solution of modified Bipro™ (from the same source as example 4; protein content=90% w/w) and 10.9% lactose was mixed with 720 grams of skim milk. The final protein concentration of this solution was 2.7%

Drinking yogurt was prepared in a comparable way as described for (drinking) yogurt A.

Despite the lower protein concentration in drinking yogurt B, the product obtained had a higher viscosity than the reference drinking yogurt A. A test panel evaluation resulted in a preference for the drinking yogurt B, based on a more pleasant mouth feel.

### Example 10

### Use of the Protein Additive of the Invention as a Foaming Agent in Meringue

The foaming capacity of the composition of the invention was tested in the preparation of meringue. Compositions were prepared according to the following table.

Composition (%)

| | Control | Invention |
|---|---|---|
| Castor sugar | 98.2 | 98.2 |
| Bipro ™ | 1.8 | |
| Bipro ™ fibrils | | 1.8 |
| Total | 100 | 100 |

The Bipro™ protein is mixed with the sugar. Then 300 g thereof is added to a grease free mixing bowl (Hobart N 50). Subsequently, 150 g cold water is added and the composition thus obtained is mixed for 1 min (speed 1) and whipped for 6 min (speed 3).

After 6 min whipping, the amount of foam of the control composition is essentially equal to the composition of the invention (FIG. 2A). The composition of the invention leads to a foam that is more stiff than the control.

Subsequently the two variants of the foam were made into a meringue by adding 130 g sugar for each 200 g composition. The foam thus obtained is poured in small quantities on grease free paper and dried in the oven for 30 minutes at 125° C. FIG. 2B shows that the addition of a protein additive of the invention leads to a better formed meringue than when untreated Bipro™ is used.

The control meringue has an overrun of 98% and a penetration of 15 mm with the light-weight measuring probe (43 g). The meringue of the invention is more firm and has an overrun of 80% and 10 mm penetration with the same measuring probe.

### Example 11

### Use of the Protein Additive of the Invention as a Foaming Agent in Dessert Applications

A solution of 3% (w/v) of Bipro™ treated according to the invention in water was whipped during 1 min. in a Hobart mixer, speed 3. The thus obtained foam has a volume of 1100% and a good stability.

To a solution of 3% (w/v) of the protein of the invention extra calcium (0.13%) was added as Ca-lactate and treated as above. The stability of the foam is the same as without the addition of calcium.

In a third experiment 10% sugar is added to the calcium containing solution of the second experiment. The mixture thus obtained is whipped in the same way resulting in an overrun of 900%. The foam is more stable than without the sugar.

The same series of experiments was performed with Bipro™ that was not treated according to the invention. In order to obtain a reasonable amount of foam the solution had to be whipped for 5 min. in the Hobart in speed 3 to achieve an overrun of 700%.

It thus follows that the treatment of the invention leads to a higher foaming ability.

In order to test the use of the treated Bipro™ of the invention as a foamig additive in desserts, a solution of 3% (w/v) of the protein of the invention and extra calcium (0.13%) was mixed with 10% sugar and 5% instant starch (Cerestar 12170) and whipped for 3 minutes. Already after 1 minute a foam was obtained but after 3 minutes the foam was firm and short with an overrun of 400%. Addition of 1% citric acid leads to an even better foam formation of about 600%.

The foaming capacity as compared to untreated Bipro™ is spectacular.

### Example 12

### Cappuccino Foamer

Recipe:

| | | | | | |
|---|---|---|---|---|---|
| A: | DP 387 | 5 g | B: | DP 387 | 5 g |
| | Powdered sugar | 3 g | | Powdered sugar | 3 g |
| | Bipro ™ | 0.5 g | | Protein of the invention | 0.5 g |

Cappuccino was made by mixing a cappuccino foamer (DP 387 from DMV International, the Netherlands), with sugar and the reference protein Bipro™ (ex. A), or the product (spray dried) of the invention (ex. B.). Subsequently 100 ml of boiling water was poured on the powder mix, and the cappuccino foam was assessed after 5 minutes.

| | Foam height | Foam appearance, taste |
|---|---|---|
| A: | 7 mm | good foam, fine structure |
| B: | 10 mm | foam with firmer body as in A; |
| | | more stable foam compared to A, |
| | | milky, frothy. |

The protein according to the invention clearly improves the foaming properties of a cappuccino foamer (FIG. 3).

1-46. (canceled)

47. A method for improving the functional properties of globular proteins, comprising the steps of:
a) providing a solution of one or more globular proteins, in which solution the protein is at least partially aggregated in fibrils; and
b) performing one or more of the following steps in random order:
i) adjusting the pH of the solution to about neutral;
ii) increasing the salt concentration in the solution;
iii) concentrating the solution;
iv) changing the solvent quality of the solution.

48. The method according to claim 47, wherein the fibril-containing solution of the one or more globular proteins is provided by heating a solution of the one or more proteins above room temperature, preferably at a temperature between about 50 and 100° C. at a pH between about 0.5 and 4.

49. The method according to claim 48, wherein the fibril-containing solution of the one or more globular proteins is provided by heating a solution of the one or more proteins above room temperature, preferably at a temperature between about 50 and 100° C. at a pH between about 0.5 and 3.

50. The method according to claim 48, wherein the solution is heated during a period of at least 10 minutes.

51. The method according to claim 48, wherein the solution is heated during a period of at least one hour.

52. The method according to claim 48, wherein the solution is heated during a period of at least 6 hours.

53. The method according to claim 48, wherein the solution is heated during a period of at least 8 hours.

54. The method according to claim 47, wherein the solution is cooled before performing one or more of steps i) to iv).

55. The method according to claim 54, wherein the solution is cooled to a temperature between denaturation temperature and 20° C.

56. The method according to claim 54, wherein the solution is cooled to a temperature between denaturation temperature and 5° C.

57. The method according to claim 48, wherein the heating is performed at a pH below 2.8, preferably below 2.5, more preferably below 2.2.

58. The method according to claim 47, wherein the fibril-containing solution of the one or more globular proteins is provided by adding a denaturing agent to the solution.

59. The method according to claim 58, wherein the denaturing agent is a hydrotropic or chaotropic agent.

60. The method according to claim 58, wherein the denaturing agent is selected from the group consisting of ureum, guanidinium chloride and alcohols, such as methanol, ethanol, propanol, butanol and trifluorethanol.

61. The method according to claim 50, wherein the solution has a pH of 0.5-14.

62. The method according to claim 48, wherein the globular protein is a protein that is substantially non-denatured and is capable of being thermally denatured at a temperature at or above the denaturation temperature of the protein or capable of being chemically denatured.

63. The method according to claim 48, further comprising the step of adding already formed fibrils to the solution prior to production of the fibril-containing solution.

64. The method according to claim 63, wherein the already formed fibrils are obtainable by the method according to claim 48.

65. The method according to claim 63, wherein the amount of already formed fibrils based on the total amount of protein is more than 0 and less than 90%.

66. The method according to claim 63, wherein the amount of already formed fibrils based on the total amount of protein is between 10 and 80%.

67. The method according to claim 63, wherein the amount of already formed fibrils based on the total amount of protein is between 20 and 70%.

68. The method according to claim 63, wherein the amount of already formed fibrils based on the total amount of protein is between 30 and 60%.

69. The method according to claim 47, wherein the pH is increased to a value between 3.9 and 9.

70. The method according to claim 47, wherein the pH is increased to a value about neutral pH.

71. The method according to claim 47, wherein the salt concentration is increased to a maximum of 0.2M.

72. The method according to claim 47, wherein the salt concentration is increased to a maximum of 0.1M.

73. The method according to claim 72, wherein the salt used for increasing the salt concentration is the salt of a divalent ion, preferably calcium.

74. The method according to claim 72, wherein the salt used for increasing the salt concentration is the salt of calcium.

75. The method according to claim 47, wherein step i) is performed prior to step ii).

76. The method according to claim 47, wherein the solvent quality of the solution is changed by removing the denaturing agent.

77. The method according to claim 47, further comprising the step of drying the solution to obtain a dry product.

78. The method according to claim 77, wherein the drying comprises spray drying.

79. The method according to claim 77, wherein the dry product is a powder.

80. The method according to claim 47, wherein the globular protein is selected from the group consisting of whey and proteins, egg albumins, blood globulins, soy proteins and wheat proteins.

81. The method according to claim 47, wherein the globular protein is selected from the group consisting of prolamines, potato proteins and pea proteins.

82. The method according to claim 80, wherein the globular protein is a whey protein isolate or a whey protein concentrate.

83. The method according to claim 80, wherein the globular protein is a whey protein concentrate enriched in β-lactoglobulin.

84. The method according to claim 83, wherein the globular protein is the whey protein isolate Bipro™.

85. The method according to claim 83, wherein the globular protein is β-lactoglobulin.

86. A protein additive based on a system of one or more proteins that are at least partially aggregated in fibrils, wherein the protein additive has improved functional properties as compared to a similar protein additive based on a system of the same one or more proteins in the same concentration in which the proteins are not aggregated in fibrils.

87. The protein additive according to claim 86, wherein the functional properties are one or more of the following: foaming properties, thickening properties, gelling properties and emulsifying properties.

88. The protein additive obtainable by the method according to claim 47.

89. The protein additive according to claim 86, wherein the protein additive is in dry form.

90. The protein additive according to claim 86 for use as a stabilizer of foams, dispersions and emulsions.

91. The protein additive according to claim 86 for use in dairy products.

92. The protein additive according to claim 86 for use in meat products.

93. The protein additive according to claim 86 for use in paints.

94. The protein additive according to claim 86 for use in toothpastes, cosmetics, deodorants.

95. A dairy product comprising the protein additive according to claim 86.

96. A meat product comprising the protein additive according to claim 86.

97. A paint comprising the protein additive according to claim 86.

98. A toothpaste comprising the protein additive according to claim 86.

99. A cosmetic comprising the protein additive according to claim 86.

100. A deodorant comprising the protein additive according to claim 86.

101. A protein composition comprising one or more particles having texturizing properties, wherein the protein molecules are aggregated into fibrils.

102. The protein composition according to claim 101, wherein the texturizing properties comprise the ability to promote or modify the viscosity or gelling ability of a product containing the composition.

103. The protein composition according to claim 101, wherein the fibrils have an aspect ratio defined as the ratio between length and width or length and height or length and diameter of 5 or higher.

104. The protein composition according to claim 101, wherein the length of the fibrils is preferably equal to or about 100 Å and equal to or below 1 μm, preferably below 100 μm.

105. The protein composition according to claim 104, wherein the length of the fibrils is preferably equal to or above 100 Å and below 100 μm.
